(19) United States
(12) Patent Application Publication
(10) Pub. No.: US 2001/0036486 A1
(43) Pub. Date: Nov. 1, 2001

(54) Method for treating nicotine addiction and deterring tobacco use with hypericum perforatum

(76) Inventors: Norman E. Rosenthal, Rockville, MD (US); Richard A. Friedman, New York, NY (US)

Correspondence Address: SHANKS & HERBERT, Transpotomac Plaza, 1033 N. Fairfax St., Suite 306, Alexandria, VA, 22314, US

(21) Appl. No.: 09/838,263

(22) Filed: Apr. 20, 2001

Related U.S. Application Data
(60) Provisional application No. 60/202,823, filed on May 8, 2000.

(51) Int. Cl. A61K 35/78

(52) U.S. Cl. 424/730; 424/734; 424/737; 424/733; 424/778

(57) ABSTRACT

A method of treating nicotine addiction or deterring tobacco use in a subject by administering to the subject a therapeutically effective amount of Hypericum perforatum or at least one active component thereof is disclosed. As disclosed, the composition may further comprise a pharmaceutically acceptable carrier. The composition may also comprise a supplementary compound such as L-phenylalanine, L-tyrosine, tryptophan, 5-hydroxy tryptamine, serotonin, calcium carbonate and magnesium oxide, kava-kava, a kava alkaloid, valerian, hops, a passion flower extract, vitamin C, or Echinacea.

### RELATED APPLICATION DATA

This application claims the benefit of U.S. Provisional Patent Application No. 60/202,823, filed May 8, 2000, naming Norman E. Rosenthal, and Richard Friedman, which is herein incorporated by reference.

### BACKGROUND OF THE INVENTION

1. Field of the Invention

The invention relates to a method of deterring, tobacco use, ending tobacco use, treating nicotine addiction, or a combination thereof in a subject by administering to the subject a composition comprising a therapeutically effective amount of Hypericum perforatum, or at least one active component thereof or both.

2. Description of the Related Art

Tobacco use is the number one source of preventable death, primarily due to cardiovascular disease and cancer, in the United States. Approximately one-half of the 50 million smokers try to quit each year, but only about 6% of those who attempt to quit succeed in the long term. See MMWR Morb Mortal Wkly Rep (1993) 42:504-507.

Nicotine replacement therapies, such as Nicorette® gum and nicotine epidermal patches, help some smokers stop smoking. Nicotine replacement therapies, however, are not effective in treating tobacco use as approximately 70-80% of those who use nicotine replacement therapies relapse. See Fiore, M. C., et al., (1996) AHCPR publication no. 96-0692. Furthermore, nicotine replacement therapies do not treat nicotine addiction as nicotine is continuously administered to the subject being treated.

Since tobacco users have significantly higher rates of depression than non-tobacco users, some antidepressants have been tested for deterring tobacco use. Administration of Wellbutrin SR (bupropion), a dopaminergic and noradrenergic antidepressant, produces longer periods of smoking cessation than both nicotine patches and placebos. See Jorneby, D. E., et al., (1999) N Engl J Med 340:685-691. However, it is arguable whether treatment with a serotonin reuptake inhibitor (SRI) has effect on smoking cessation. See Sellers, Em, et al., (1987) J Clin Psycopharmocol. 7:417-420, but cf., Koch, D. J., et al., U.S. Pat. No. 5,912,256. In a small study, doxepin, a tricyclic antidepressant, showed some promise for helping smoking cessation. See Edward, N. B., et al., (1989) Am J Psychiatry 146:373-376. Unfortunately, no follow-up large trials were conducted. Although doxepin enhances serotonin neurotransmission, it is unclear whether serotonin is involved in smoking cessation. Thus, whether doxepin has an effect on smoking cessation is unknown.

Nicotine elevates the levels of dopamine, norepinephrine and other neurotransmitters in the key areas of the brain which are involved in the reinforcement effects of drugs such as cocaine, amphetamines, and opiates. Bupropion blocks the neuronal reuptake of dopamine and norepinephrine, thereby increasing dopamine activity in those areas of the brain that mediate the reinforcing effects of addictive drugs like nicotine, which may account for its efficacy in smoking cessation.

However, bupropion and other prescription medications can have serious side effects which include dependence, seizures, nausea, headaches, constipation and dizziness. Furthermore, not only are these prescription medications expensive, but one is required to see a doctor in order to obtain a prescription. Consequently, the cost involved in obtaining a prescription and the potential embarrassment of having to acknowledge the addiction and the need for help, may deter an individual from seeking help. Therefore, a need remains for an effective treatment for nicotine addiction and/or deterring tobacco use which is non-addictive, inexpensive, easy to obtain over-the-counter and lacks serious side effects.

Hypericum perforatum, also known as St. John's wort, has been used to treat major depression, dysthymia, seasonal affective disorder, obsessive compulsive disorder, panic disorder, social phobia, subsyndromal depression, trichotillomania, stress, insomnia, and premenstrual syndrome. See Rosenthal, N. E., (1998) ST. JOHN's WORT: THE HERBAL WAY TO FEEL GOOD. Harper Collins. Hypericum perforatum contains many bioactive compounds, such as hypericin, hyperforin, flavonoids and xanthone derivatives, which are believed to be effective in treating depression. See Rosenthal, N. E., (1998) ST. JOHN'S WORT: THE HERBAL WAY TO FEELING GOOD. Harper Collins; and Linde, K. G. et al., (1996) British Med. J. 313:253-258. Hypericum perforatum inhibits the reuptake of dopamine and norepinephrine and serotonin like bupropion and SRI's, but does not cause serious side effects like the prescription antidepressants. See Muller W. E., et al., (1997) Pharmacopsychiatry 30 (suppl. 2). In addition, Hypericum perforatum has been found to affect gamma aminobuytric acid (GAMA) receptors, which are known to mediate calming, anti-anxiety responses which may be useful to combat the anxiety associated with nicotine withdrawal. According to one German study, of 3250 subjects taking Hypericum perforatum, only 2.4% of subjects reported side effects and only 1.5% stopped taking Hypericum perforatum due to the side-effects. See Woelk, H. et al., (1994) J Geriatr Psychiatry Neurol 7(Suppl. 1):34-38

Recently, the applicants have discovered that Hypericum perforatum is effective in deterring or ceasing tobacco use, treating nicotine addiction or cravings, or a combination thereof. Therefore, methods of treating nicotine addiction and deterring tobacco use by the administration of Hypericum perforatum and/or at least one active component thereof are disclosed herein below.

### SUMMARY OF THE INVENTION

The invention is directed to a method of treating nicotine addiction in a subject comprising administering to the subject a composition comprising a therapeutically effective amount of Hypericum perforatum or at least one active component thereof or both.

In another embodiment, the invention is directed to a method of deterring tobacco use by a subject comprising administering to the subject a composition comprising a therapeutically effective amount of Hypericum perforatum or at least one active component thereof or both.

In some embodiments, the composition may comprise a pharmaceutically acceptable carrier. In some embodiments, the composition is orally administered in about 1-4 single doses per day. In other embodiments, administration may be intravenous, intradermal, subcutaneous, transdermal, transmucosal, or rectal.

In some embodiments of the invention, the therapeutically effective amount of Hypericum perforatum or the active component thereof is about 1 mg to about 1800 mg, preferably about 50 to about 1800 mg.

In one embodiment, the total daily amount of Hypericum perforatum of the active component thereof administered to the subject is about 1 mg to about 1800 mg, preferably about 50 mg to about 1800 mg per day.

In a preferred embodiment, the total daily amount of Hypericum perforatum or the active component thereof administered to the subject is about 0.001 mg to about 50 mg, preferably about 1 mg to about 50 mg per kg of the subject being treated, more preferably about 25 mg to about 30 mg per kg of the subject being treated.

In some embodiments of the invention, the composition further comprises a supplementary compound such as L-phenylalanine, L-tyrosine, tryptophan, 5-hydroxy tryptamine, serotonin, calcium carbonate, magnesium oxide, kava-kava, kava alkaloids, valerian, hops, passion flower extract, vitamin C, Echinacea, agents which may help buffer Hypericum perforatum or the active component thereof, and agents which may act synergistically with Hypericum perforatum or the active component thereof.

In one embodiment, the invention is directed to a method of treating nicotine addiction and deterring tobacco use in a subject comprising administering to the subject a composition comprising a therapeutically effective amount of Hypericum perforatum or at least one active component thereof or both.

In some embodiments, the composition comprising a therapeutically effective amount of Hypericum perforatum or at least one active component thereof or both is administered about two weeks prior to ending the subject's exposure to nicotine or ending the subject's tobacco use.

In some embodiments, the composition comprising a therapeutically effective amount of Hypericum perforatum or at least one active component thereof or both is administered for up to about 8 weeks after ending the subject's exposure to nicotine or ending the subject's tobacco use.

In some embodiments, the composition comprising a therapeutically effective amount of Hypericum perforatum or at least one active component thereof or both is administered indefinitely after ending the subject's exposure to nicotine or ending the subject's tobacco use.

### DETAILED DESCRIPTION OF THE INVENTION

The present invention relates to a method of treating nicotine addiction and deterring tobacco use in a subject by the administration of Hypericum perforatum or at least one active component thereof or both. The active components in Hypericum perforatum include hyperforin, hypericum, flavonoids, flavanoid derivatives such as rutin and hyperin, hypericine, pseudo-hypericine, xanthone derivatives, amentoflavone, biapigenin, hypericum oil, hypericin and pseudohypericin.

Hypericum perforatum inhibits serotonin uptake by synaptosomes, and thereby increases serotonin levels in an individual. The antidepressant activity of Hypericum perforatum is due to the elevated serotonin levels or its effects on norepinephrine and dopamine. See Perovic, et al., (1996) Drug Research. 45(11):1145-1148. Hypericum perforatum is reported to have no serious side effects or produces only minor side effects, such as gastrointestinal symptoms, allergic reactions, and fatigue. See Harrer, et al., (1994) Phytomedicine 1:3-8; and De Smet, et al., (1996) BMJ London. Although Hypericum perforatum is known for use as an antidepressant, it has not been used for treating nicotine addiction or promoting tobacco use cessation or both. Therefore, the present invention provides a method for treating nicotine addiction and/or deterring tobacco use by the administration of Hypericum perforatum, or at least one active component thereof or both.

As used herein, “tobacco use” refers to, but is not limited to, cigarette smoking, cigar smoking, smoking other tobacco and products containing nicotine, and chewing tobacco and other products containing nicotine or derivatives thereof or nicotine-like substances.

“Nicotine addition” includes, but is not limited to, nicotine addiction induced by tobacco, nicotine patch and nicotine gum use.

Hypericum perforatum, or at least one active component thereof or both may be incorporated into a pharmaceutical composition suitable for administration. Such composition typically comprises Hypericum perforatum, hyperforin, hypericum, flavonoids, flavanoid derivatives such as rutin and hyperin, hypericine, pseudo-hypericine, xanthone derivatives, amentoflavone, biapigenin, hypericum oil, hypericin or pseudohypericin or a combination thereof and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated.

Supplementary active compounds can also be incorporated into the compositions. Supplementary active compounds include compounds which affect the neurotransmitters and receptors involved in nicotine addiction, depression or both. For example, L-phenylalanine is considered to be a supplementary active compound as it is the precursor of tyrosine which is required for the synthesis of the neurotransmitters epinephrine, norepinephrine and dopamine. Other supplementary active compounds include L-tyrosine, tryptophan, 5-hydroxy tryptamine, serotonin, calcium carbonate, magnesium oxide, kava-kava, kava alkaloids, valerian, hops, passion flower extract, vitamin C, Echinacea, agents which may help buffer Hypericum perforatum or the active component thereof, and agents which may act synergistically with Hypericum perforatum or the active component thereof.

The pharmaceutical composition of the invention is formulated to be compatible with its intended route of administration. Hypericum perforatum formulations may be prepared by methods known in the art and as set forth in St. John's Wort monographs. See, for example, Remington's Pharmaceutical Sciences 18^{th} ed (1990) E. W. Martin ed. Mack Publishing Co. Pa., and see The United States Pharmacopoeia (USP 24) and The National Formulary (NF 19). Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. The pH of the solutions or suspensions can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. The composition must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol (PEG), and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating Hypericum perforatum, or at least one active component thereof or both in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration of the compounds can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

Pharmaceutical compositions for oral, intranasal, or topical administration can be supplied in solid, semi-solid or liquid forms, including tablets, capsules, powders, liquids, and suspensions. For administration via the respiratory tract, a preferred composition is one that provides a solid, powder, or liquid aerosol when used with the appropriate aerosolizer device.

In one embodiment, Hypericum perforatum, or at least one active component thereof or both can be prepared with carriers that will protect against rapid elimination from the body, such as controlled release formulations which include implants and microencapsulated delivery systems. Biodegradable, biocompatible polymers, such as ethylene vinyl acetate, polyanhydrides, polyglycolic acid, collagen, polyorthoesters, and polylactic acid, may be used. Methods for preparation of such formulations will be apparent to those skilled in the art. The materials mentioned above can also be obtained commercially from Alza Corporation and Nova Pharmaceuticals, Inc. Liposomal suspensions can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein refers to physically discrete units suited as unitary dosages for the subject to be treated. For example, each unit may contain a predetermined quantity of Hypericum perforatum or the active components thereof, which predetermined quantity is calculated to produce the desired therapeutic effect in association with the required pharmaceutical carrier.

By methods known to those skilled in the art, one may calculate the predetermined quantity which is effective in treating nicotine addiction and/or deterring tobacco use and adjust the dosage for an individual subject. The efficacy of particular dosages may be determined by (1) the extent of tobacco and nicotine cravings, (2) physical symptoms such as queasiness, tremulousness and palpitations; (3) emotional symptoms such as depression, anxiety and irritability, (4) nicotine and tobacco use, and (5) the use of other substances, such as stimulants, depressants, or any increased food intake to compensate for lack of tobacco and nicotine use. This criteria may be objectively determined by use of the Fagerstrom Tolerance Questionnaire, the Hamilton Anxiety and Depression Scales, the Beck Depression Inventory, or a combination thereof. The Fagerstrom Tolerance Questionnaire is an objective instrument which has been used to assess the extent of a subject's nicotine addiction. See Fagerstrom, K. O., (1978) Addict Behav 3:234-41.

Hypericum perforatum, at least one active component thereof, or both may prepared for addition to a composition to form the pharmaceutical composition by any suitable method known in the art. For example, Hypericum perforatum may be dried and then ground up and placed in a capsule or added to a composition. Hypericum perforatum may also be prepared by alcohol extraction wherein the flowering and leafy portions of Hypericum perforatum are dried and then dissolved in alcohol. When the alcohol evaporates, an extract containing the active components of Hypericum perforatum remain. This extract may then be administered directly or added to a composition. Alternatively, Hypericum perforatum, at least one active component thereof, or both may be prepared in the form of a pill or tincture or tea by standard methods known in the art. If an extract is formed, the Hypericum perforatum extract is standardized to contain about 10% or less hypericin.

As defined herein, a therapeutically effective amount of Hypericum perforatum or the active components thereof (i.e., an effective dosage) ranges from about 0.001-50 mg of Hypericum perforatum per kg of body weight, preferably about 1-50 mg of Hypericum perforatum per kg of body weight, more preferably about 10-40 mg of Hypericum perforatum per kg of body weight, and even more preferably about 20-30 mg of Hypericum perforatum per kg of body weight. The skilled artisan will appreciate that certain factors may influence the dosage required to effectively treat a subject, including but not limited to, the severity the nicotine addiction or tobacco use, previous treatments, the general health and age of the subject, and other diseases present.

In a preferred example, a subject is treated with a composition comprising Hypericum perforatum, or at least one active component thereof or both in the range of between about 0.001-50 mg of Hypericum perforatum per kg of body weight, about one to about three times a day for about 1-10 weeks, preferably for about 2-8 weeks, more preferably for about 3-7 weeks, and even more preferably for about 4-6 weeks. However, one of ordinary skill in the art will appreciate that the length of treatment will vary depending on the degree of nicotine addition and will therefore adjust the treatment regime accordingly. For example, some patients may require treatment indefinitely to remain abstinent. It will also be appreciated that the effective dosage of Hypericum perforatum or the active components thereof used for treatment may increase or decrease over the course of a particular treatment.

The pharmaceutical compositions can be included in a container, pack, or dispenser together with instructions for administration.

The following examples are intended to illustrate but not to limit the invention.

### EXAMPLE 1

### Clinical Treatment for Nicotine Addiction

A. Case Study 1

A 57 year old divorced white female subject with a history of recurrent major depression and smoking about 1.5 packs of cigarettes per day for approximately 28 years, tried numerous times, without success, to quit smoking with the aid of various nicotine replacement products. She would normally relapse within about 3-8 weeks.

She was not depressed at the time of treatment with Hypericum perforatum, and had been in remission from depression for at least 2 years. She began taking 300 mg of Hypericum perforatum twice a day for 1 week and then 300 mg of Hypericum perforatum three times a day for the second week before quitting smoking. During the first week, her craving for nicotine was strong, but much less intense than during previous attempts to quit. Craving was measured by use of the Fagerstrom Tolerance Questionnaire. Treatment with 600 mg of Hypericum perforatum two times a day was continued for about 8 weeks. She remained continuously abstinent for about 7 months since the start of treatment with Hypericum perforatum. She had a brief relapse at about 7 months for about 2 weeks, but resumed and remained abstinence without any further Hypericum perforatum treatment beyond the initial 8 weeks of Hypericum perforatum treatment.

B. Case Study 2

A 48 year old married white male with a 30 year history of smoking about 1-1.5 packs of cigarettes per day. He had no known psychiatric history. He tried quitting many times, but was unable to maintain abstinence for greater than about 1 month. He began taking 300 mg of Hypericum perforatum twice a day for week one and then took 300 mg of Hypericum perforatum three times a day for the second week before quitting smoking. Craving was measured by use of the Fagerstrom Tolerance Questionnaire. He had only very mild nicotine craving and remains abstinent.

### EXAMPLE 2

### Efficacy of Hypericum perforatum in Treating Nicotine Addiction and Promoting Tobacco Use Cessation

In order to establish efficacy definitively, a double blind test may be performed. This may be done by administering Hypericum perforatum for about one week to about three months versus a placebo in a double blind design. Hypericum perforatum may be administered in dosages of about 300 mg three times a day, the modal dosage used for the treatment of depression, and placebo may be given at the same time.

Alternatively, smaller doses of Hypericum perforatum, going all the way down to about 100 mg per day or larger doses, going all the way up to about 1800 mg per day (the largest dosage used thus far in depression trials) could be administered.

Another study to determine the efficacy of Hypericum perforatum, would be a randomized double-blind clinical trial comparing various doses of Hypericum perforatum with various doses of bupropion, and placebos.

### EXAMPLE 3

### Measurement of Cravings and Physical and Emotional Symptoms

The Fagerstrom Tolerance Questionnaire, Hamilton Anxiety and Depression Scales and the Beck Depression Inventory are used to objectively measure and standardize a subject's nicotine cravings, physical symptoms and emotional symptoms. These and other instruments and tests known in the art may be used. Methods of using these instruments and tests are known in the art and one of ordinary skill in the art may modify and adjust the instruments and tests as needed.

Additionally, biological samples obtained from a subject may be assayed to detect and measure the amount of stress hormones such as cortisol and norepinephrine and compare to the amounts found in biological samples obtained from normal individuals in order to determine the efficacy of the treatment, dosage, or both. The biological samples include plasma, blood, saliva, breast milk, urine and tissue samples. Standard assays, such as ELISA or RIA, may be used to detect and measure the amounts of a stress hormone.

For example, urinary cortisol may be detected and measured as described in U.S. Pat. No. 5,910,575. Cortisol found in wound fluids may be detected and measured as described in U.S. Pat. No. 5,912,114. Cortisol may be detected and measured by the cortisol radioimmunoassay method as described in U.S. Pat. Nos. 4,277,460 and 4,190,593. Norepinephrine may be detected and measured according to the radioenzymatic assay as described in U.S. Pat. No. 4,649,107.

To the extent necessary to understand or complete the disclosure of the present invention, all publications, patents, and patent applications mentioned herein are expressly incorporated by reference therein to the same extent as though each were individually so incorporated.

What is claimed is:

1. A method of treating nicotine addiction or craving in a subject comprising administering to the subject a composition comprising a therapeutically effective amount Hypericum perforatum or at least one active component thereof.

2. The method of claim 1, wherein the composition comprises Hypericum perforatum, hyperforin, hypericum, a flavonoids or derivative thereof, a xanthone derivative, amentoflavone, biapigenin, hypericum oil, hypericin, pseudohypericin, hypericine, pseudo-hypericine, or a combination thereof.

3. The method of claim 1, wherein the composition further comprises a pharmaceutically acceptable carrier.

4. The method of claim 1, wherein the composition is administered in an amount of about 1 to about 3 single doses per day.

5. The method of claim 4, wherein the composition is administered in an amount of 3 single doses per day.

6. The method of claim 1, wherein the therapeutically effective amount is about 0.001 mg to about 50 mg per kg of the subject.

7. The method of claim 6, wherein the therapeutically effective amount is about 1 mg to about 50 mg per kg of the subject.

8. The method of claim 7, wherein the therapeutically effective amount is about about 25 mg to about 30 mg per kg of the subject.

9. The method of claim 1, wherein the composition is administered intravenously, intradermally, subcutaneously, orally, transdermally, transmucosally, or rectally.

10. The method of claim 1, wherein the composition is in a tablet or a capsule form.

11. The method of claim 1, wherein the composition further comprises at least one supplementary active compound.

12. The method of claim 11, wherein the supplementary active compound is L-phenylalanine, L-tyrosine, tryptophan, 5-hydroxy tryptamine, serotonin, calcium carbonate and magnesium oxide, kava-kava, a kava alkaloid, valerian, hops, a passion flower extract, vitamin C, or Echinacea.

13. The method of claim 1, wherein the subject is administered about 1 mg to about 1800 mg per day of Hypericum perforatum or at least one active component thereof.

14. A method of deterring or ending tobacco use by a subject comprising administering to the subject a composition comprising a therapeutically effective amount Hypericum perforatum or at least one active component thereof.

15. The method of claim 14, wherein the composition comprises Hypericum perforatum, hyperforin, hypericum, a flavonoids or derivative thereof, a xanthone derivative, amentoflavone, biapigenin, hypericum oil, hypericin, pseudohypericin, hypericine, pseudo-hypericine, or a combination thereof.

16. The method of claim 14, wherein the composition further comprises a pharmaceutically acceptable carrier.

17. The method of claim 14, wherein the composition is administered in an amount of about 1 to about 3 single doses per day.

18. The method of claim 14, wherein the composition is administered in an amount of 3 single doses per day.

19. The method of claim 14, wherein the therapeutically effective amount is about 0.001 mg to about 50 mg per kg of the subject.

20. The method of claim 19, wherein the therapeutically effective amount is about 1 mg to about 50 mg per kg of the subject.

21. The method of claim 20, wherein the therapeutically effective amount is about about 25 mg to about 30 mg per kg of the subject.

22. The method of claim 14, wherein the composition is administered intravenously, intradermally, subcutaneously, orally, transdermally, transmucosally, or rectally.

23. The method of claim 14, wherein the composition is in a tablet or a capsule form.

24. The method of claim 14, wherein the composition further comprises at least one supplementary active compound.

25. The method of claim 24, wherein the supplementary active compound is L-phenylalanine, L-tyrosine, tryptophan, 5-hydroxy tryptamine, serotonin, calcium carbonate and magnesium oxide, kava-kava, a kava alkaloid, valerian, hops, a passion flower extract, vitamin C, or Echinacea.

26. The method of claim 14, wherein the subject is administered about 1 mg to about 1800 mg per day of Hypericum perforatum or at least one active component thereof.

27. A method of treating nicotine addiction or deterring tobacco use in a subject comprising administering to the subject a composition comprising a therapeutically effective amount Hypericum perforatum or at least one active component thereof.

28. The method of claim 27, wherein the composition is administered about two weeks prior to ending the subject's exposure to nicotine or tobacco use.

29. The method of claim 27, wherein the composition is administered for up to about 8 weeks after ending the subject's exposure to nicotine or tobacco use.

30. The method of claim 27, wherein the composition is administered indefinitely after ending the subject's exposure to nicotine or tobacco use.
